(19) United States
(12) Patent Application Publication
(10) Pub. No.: US 2022/0111104 A1
(43) Pub. Date: Apr. 14, 2022

(54) INTELLIGENT FULL-TIME STERILIZATION LIGHTING DEVICE

(71) Applicants: SHIH FONG LIN, Taoyuan (TW); WU YI HSU, Taoyuan (TW)

(72) Inventors: SHIH FONG LIN, Taoyuan (TW); WU YI HSU, Taoyuan (TW)

(21) Appl. No.: 17/070,607

(22) Filed: Oct. 14, 2020

(51) Int. Cl. A61L 9/20

(52) U.S. Cl. CPC A61L 9/20; A61L 2209/12; A61L 2209/111

(57) ABSTRACT

An intelligent full-time sterilization lighting device is disclosed. It contains an external casing, a plurality of ultraviolet sterilization light source, an airflow driving module and control module. Based on the designs of the air channel portion, the air inlet portion and the air outlet portion external casing, it is allowed to hide the ultraviolet sterilization light source in the air channel portion. It can sterilize the airflow introduced by the airflow driving module without harming the surrounding people. Indoor lighting can be provided under the external casing. In addition, the shape of the external casing can be designed according to the size of the calcium silicate board of the existing light steel frame. It is easy to install on light steel frame.

### FIELD OF THE INVENTION

The present invention relates to an illuminating light device. More particularly, the present invention relates to an intelligent full-time sterilization lighting device which can be installed on the existing light steel frame ceiling.

### BACKGROUND OF THE INVENTION

Ultraviolet light has a great disinfection and sterilization effect on bacteria and viruses harmful to the human body. The principle of disinfection and sterilization is that after bacteria and viruses are irradiated with ultraviolet light, the structure of DNA and RNA at the core of their lives will be directly destroyed by ultraviolet light, therefore, the protein needed for survival or reproduction cannot be formed, causing immediate death or loss of reproduction ability of bacteria and viruses. Generally speaking, the disinfection and sterilization effect can be achieved within 1 to 2 seconds after ultraviolet light irradiation. However, there is a good side and a bad side to all things. Ultraviolet light can eliminate bacteria and viruses that are harmful to humans, and it can also cause harm to humans. For example, under ultraviolet light exposure, human skin will gradually produce melanin. If the duration and frequency of exposure continue to increase, it may lead to melanoma. Ultraviolet light can damage the lens of the eye, easily causing skin cancer around the eye and deterioration and degeneration of the retina, and in severe cases, it may damage the transparency of the lens and cause blindness. Therefore, when using ultraviolet light to disinfect the surrounding environment, it should pay special attention to safety.

On the other hand, due to the recent raging Covid-19, people are thinking about how to maintain the quality of indoor air, especially how to disinfect and sterilize indoors that are almost closed. Since many buildings are equipped with light steel frame ceilings in the internal space, if ultraviolet light can be introduced for sterilization but the sterilization operation is on the light steel frame ceiling, in addition to avoiding the aforementioned unfavorable conditions for people, it can also circulate the airflow for a long time to disinfect and sterilize the indoor space. However, there is no relevant and suitable solution right now.

### SUMMARY OF THE INVENTION

This paragraph extracts and compiles some features of the present invention; other features will be disclosed in the follow-up paragraphs. It is intended to cover various modifications and similar arrangements included within the spirit and scope of the appended claims.

In order to solve the aforementioned problems, an intelligent full-time sterilization lighting device is disclosed. It comprises: an external casing, formed of an air channel portion and an air inlet portion and an air outlet portion on both sides thereof, wherein a lighting zone is formed below an outside of the air channel portion, and two sides of the lighting zone in correspondence to the air inlet portion and the air outlet portion form an air inlet zone and an air outlet zone, respectively, wherein at least one illuminating light is installed in the lighting zone, the air inlet zone has at least one air inlet, the at least one air inlet is equipped with an air filter to filter floating dust in the external air, the air outlet zone has at least one air outlet, the at least one air outlet is equipped with an air filter to filter floating dust in the air flowing through the external casing, and an angle between a normal direction of the lighting zone and a normal direction of the at least one air inlet and an angle between the normal direction of the lighting zone and a normal direction of the at least one air outlet are not larger than a first angle; a plurality of ultraviolet sterilization light sources, installed in the air channel portion; an airflow driving module, installed on any location of the air channel portion, the air inlet portion or the air outlet portion, initiated to generate an airflow from the at least one air inlet to the at least one air outlet through the air channel portion; and a control module, electrically connected to the illuminating light, the ultraviolet sterilization light sources and the airflow driving module, controlling on and off of the electrically connected components.

Preferably, the first angle is 10 degrees.

According to the present invention, the air inlet portion may form a curved segment, connected to the air channel portion and the at least one air inlet to reduce noise when airflow passes by.

According to the present invention, the air outlet portion may form a curved segment, connected to the air channel portion and the at least one air outlet to reduce noise when airflow passes by.

Preferably, the ultraviolet sterilization light source may be an ultraviolet LED light bar, a strip ultraviolet sterilization lamp or a circular ultraviolet sterilization lamp.

The control module may further comprise: a power conversion unit, electrically connected to a power switch externally, wherein when the power switch is on, AC power from the power switch is received and voltage thereof is dropped and converted to obtain a working power; an operating interface, connecting to an operating device externally; and a control unit, electrically connected to the power conversion unit, the illuminating light, the ultraviolet sterilization light sources and the airflow driving module and signally connected to the operating interface, executing the following operations: receiving a long-time and full-field operating signal from the operating device so as to keep turning on the illuminating light, the ultraviolet sterilization light sources and the airflow driving module; and receiving a short-time and half-field operating signal from the operating device so as to turn off the illuminating light, and keep turning on the ultraviolet sterilization light sources and the airflow driving module at least one operating time. The operating time may be one hour.

The control module may further comprise a communication unit, signally connected to the control unit, wherein the communication unit is able to signally connect to communication units in other intelligent full-time sterilization lighting devices to transmit operating signals from the operating device to signally connected intelligent full-time sterilization lighting devices, or synchronize operations of all signally connected intelligent full-time sterilization lighting devices.

Preferably, the operating interface is a USB connector, a RJ10 connector, a RJ45 connector, an infrared signal transceiver module or a mobile communication module.

Preferably, the communication unit may be a power line contact.

In one embodiment, the ultraviolet sterilization light sources are closer to the air outlet portion while the airflow driving module is closer to the air inlet portion.

Preferably, the airflow driving module may be a centrifugal blower, an axial blower, a crossflow blower or a combination of a plurality of fans.

Based on the designs of the air channel portion, the air inlet portion and the air outlet portion external casing, it is allowed to hide the ultraviolet sterilization light source in the air channel portion. It can sterilize the airflow introduced by the airflow driving module without harming the surrounding people. Indoor lighting can be provided under the external casing. In addition, the shape of the external casing can be designed according to the size of the calcium silicate board of the existing light steel frame. It is easy to install on light steel frame.

### BRIEF DESCRIPTION OF THE DRAWINGS

FIG. 1 is a schematic diagram of the appearance of an intelligent full-time sterilization lighting device according to an embodiment of the present invention.

FIG. 2 is a schematic diagram of the intelligent full-time sterilization lighting device viewed downward along the AA′ section in FIG. 1.

FIG. 3 is a schematic diagram of the intelligent full-time sterilization lighting device viewed upward along the section AA′ in FIG. 1.

FIG. 4 shows an embodiment of the airflow driving module in another different position.

FIG. 5 shows an embodiment of the airflow driving module in still another different position.

FIG. 6 shows an embodiment of the airflow driving module in still another different position.

FIG. 7 is a circuit diagram of the control module of the intelligent full-time sterilization lighting device.

FIG. 8 is a schematic diagram of the appearance of another intelligent full-time sterilization lighting device according to an embodiment of the present invention.

### DETAILED DESCRIPTION OF THE PREFERRED EMBODIMENTS

The present invention will now be described more specifically with reference to the following embodiments.

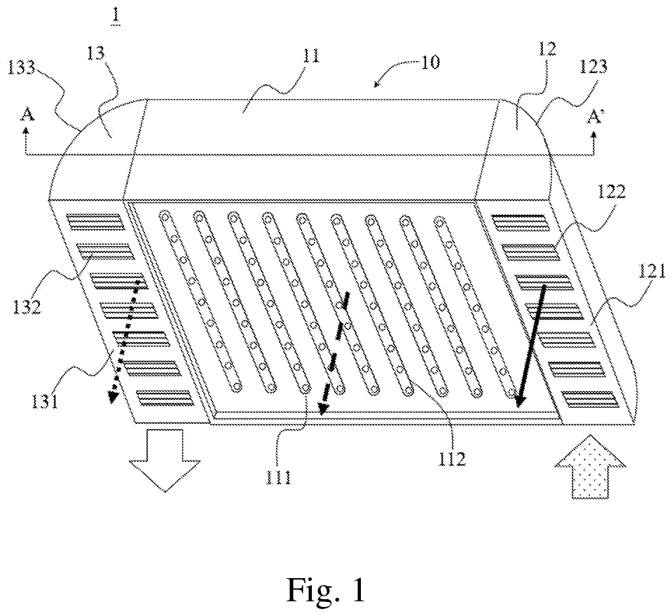

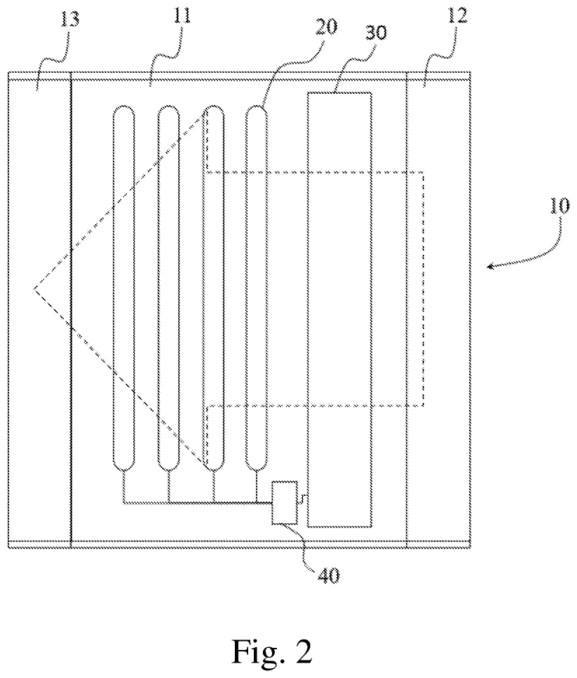

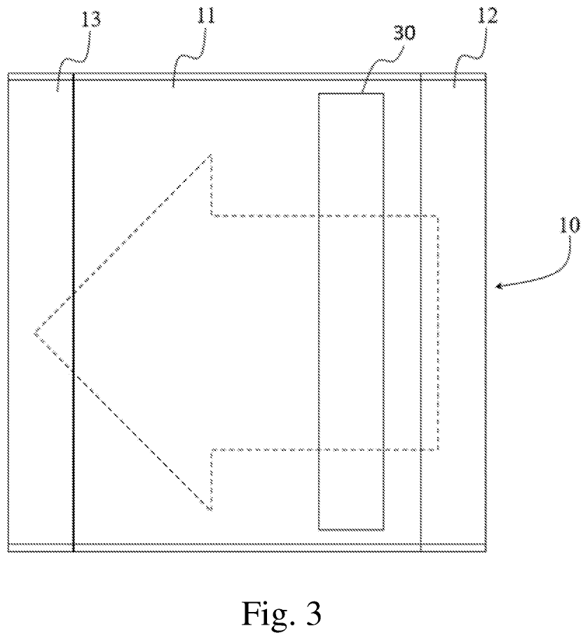

Please refer to FIG. 1 to FIG. 3. FIG. 1 is a schematic diagram of the appearance of an intelligent full-time sterilization lighting device 1 according to an embodiment of the present invention. FIG. 2 is a schematic diagram of the intelligent full-time sterilization lighting device 1 viewed downward along the AA′ section in FIG. 1. FIG. 3 is a schematic diagram of the intelligent full-time sterilization lighting device 1 viewed upward along the section AA′ in FIG. 1. According to the present invention, the intelligent full-time sterilization lighting device 1 comprises an external casing 10, a plurality of ultraviolet sterilization light sources 20, an airflow driving module 30 and a control module 40. The structure, function and interaction of the aforementioned technical components with other technical components will be detailed below.

The external casing 10 is formed of an air channel portion 11 and an air inlet portion 12 and an air outlet portion 13 on both sides of the air channel portion 11. In the present embodiment, viewed from the bottom of the external casing 10, its shape is square with a side length of about 60 cm, which is close to the size of the calcium silicate board on the light steel frame ceiling. Hence, the intelligent full-time sterilization lighting device 1 can be used to replace a calcium silicate board and be installed in the existing light steel frame structure. No additional fixed facilities are required. The section of the air channel portion 11 is rectangular. It is a flat tubular structure. A lighting zone 111 is formed below the outer side. Two sides of the lighting zone 111 in correspondence to the air inlet portion 12 and the air outlet portion 13 form an air inlet zone 121 and an air outlet zone 131, respectively. At least one illuminating light 112 is installed in the lighting zone 111. Here, 9 groups of LED strips are used as the illuminating lights 112. In other embodiments, the number of illuminating lights 112 is not limited to 9, it can be more or only one. In terms of type, the illuminating light 112 can also be incandescent bulbs (tubes), fluorescent bulbs (tubes), halogen bulbs (tubes), etc. The invention is not limited.

The air inlet zone 121 has at least one air inlet 122. In this embodiment, the number of the air inlet 122 is 7. In practice, the number of the air inlet 122 is set according to actual demand. It is not limited to 7. The shape of the air inlet 122 is not limited to rectangle as shown in FIG. 1. It can be round, square, oval, or even regular or irregular line shapes. The at least one air inlet 122 is equipped with an air filter (inside the air inlet 122 and represented by horizontal lines) to filter floating dust in the external air. Similarly, the air outlet zone 131 has at least one air outlet 132. The number of the air outlet 132 is set according to actual needs; it is also 7 in this embodiment. The shape of the air outlet 132 is the same as that of air inlet 122. The at least one air outlet 132 is equipped with an air filter (inside the air outlet 132 and represented by horizontal lines) to filter floating dust in the air flowing through the external casing 10. Here, when the intelligent full-time sterilization lighting device 1 is initiated, it will guide the air flow to allow external air to flow into the air channel portion 11 for disinfection and sterilization. The direction of air circulation comes from the air inlet 122 in FIG. 1, following the direction of the dotted arrow to enter the air inlet portion 12. After passing through the air channel portion 11, the air comes to the air outlet portion 13. The air leaves the intelligent full-time sterilization lighting device 1 from the air outlet 132 along the direction of the blank arrow frame. Since both the air inlet 122 and the air outlet 132 are equipped with air filters, the dust in the air circulating out of the intelligent full-time sterilization lighting device 1 will be greatly reduced.

A feature of the present invention is that the direction of the air inlet 122 and the direction of the air outlet 132 are not necessarily parallel to the lighting zone 111. In terms of technical characteristics, it is that an angle between a normal direction of the lighting zone 111 (as shown by the dashed arrow in FIG. 1) and a normal direction of the at least one air inlet 122 (as shown by the solid arrow in FIG. 1) and an angle between the normal direction of the lighting zone 111 and a normal direction of the at least one air outlet 132 (as shown by the dotted arrow in FIG. 1) are not larger than a first angle. Namely, when the first angle is 0 degree, the normal direction of the air inlet 122, the normal direction of the air outlet 132 and the normal direction of the lighting zone 111 are parallel (as illustrated by FIG. 1). The airflow entering from the air inlet 122 and then coming into the air channel portion 11 requires a turn close to 90 degrees, and the airflow out of the air outlet 132 from the air channel portion 11 also requires a turn close to 90 degrees. It will easily cause wind shear noise. If the first angle is 10 degrees, the air flow only needs to be turned 80 degrees. The noise will be reduced as well. However, the first angle does not need to be set too large. It will increase the area of the air inlet portion 12 and the air outlet portion 13 and reduce the length of the air channel portion 11. Since the air channel portion 11 is a space for installing disinfection and sterilization equipment, its length reduction means that the disinfection and sterilization ability is weakened.

In addition, in order to reduce the noise caused by wind shear, the air inlet portion 12 may form a curved segment 123. The curved segment 123 is connected to the air channel portion 11 and the at least one air inlet 122 to reduce noise when airflow passes by. Similarly, the air outlet portion 13 may also form a curved segment 133. The curved segment 133 is connected to the air channel portion 11 and the at least one air outlet 132 to reduce noise when airflow passes by.

The ultraviolet sterilization light sources 20 are installed in the air channel portion 11. Its location is shown in FIG. 2. In this embodiment, 4 ultraviolet sterilization light sources 20 are used and installed on the bottom of the air channel portion 11, so it is not shown in FIG. 3. According to the present invention, the number of the ultraviolet sterilization light sources 20 is not limited. The form of the ultraviolet sterilization light source 20 can be, but not limited to an ultraviolet LED light bar, a strip ultraviolet sterilization lamp or a circular ultraviolet sterilization lamp. In this embodiment, a strip ultraviolet sterilization lamp is taken as an example to illustrate. When the air flows in the direction indicated by the dashed arrow frame in FIG. 2, the ultraviolet sterilization light source 20 that is turned on will sterilize it. Meanwhile, Because the ultraviolet light will only radiate in the air channel portion 11 (external casing 10), it will not harm the crowd outside (in a closed room).

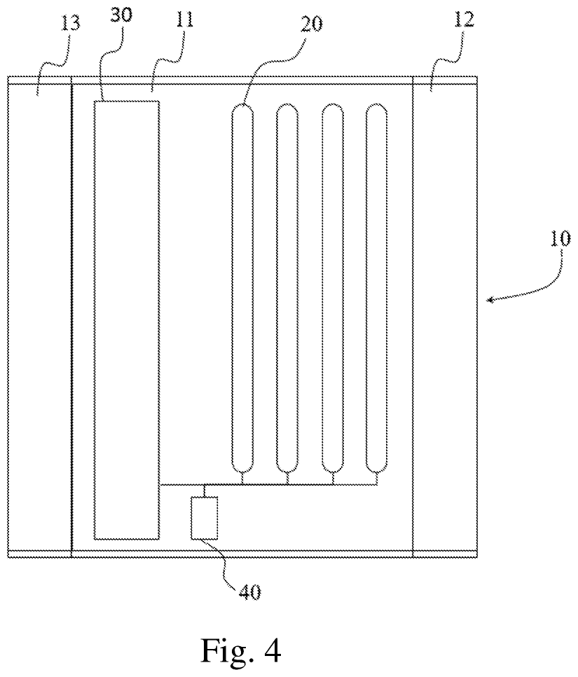

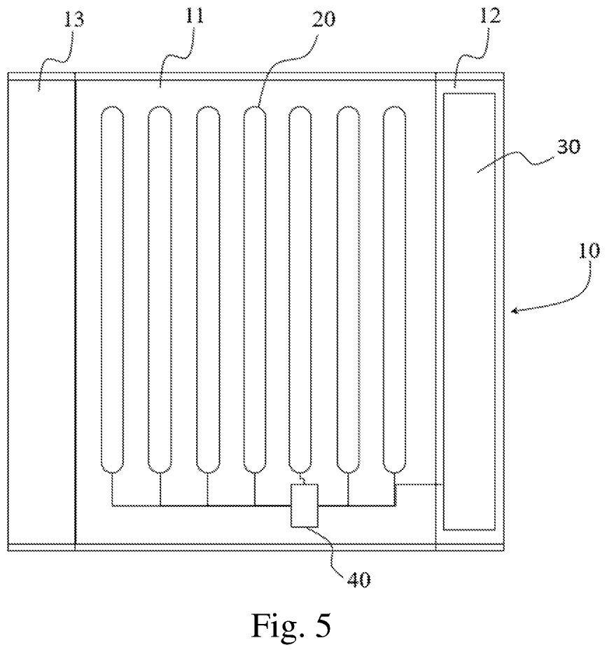

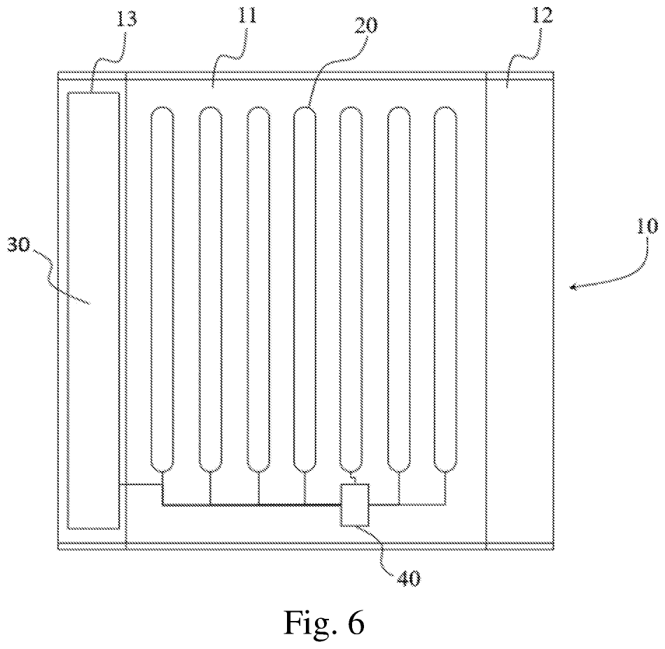

The airflow driving module 30 can be installed on any location of the air channel portion 11, the air inlet portion 12 or the air outlet portion 13 as long as the designed size permits. In the present embodiment, the airflow driving module 30 is installed in the air channel portion 11, located on an upwind side of the ultraviolet sterilization light sources 20. In other embodiments, arrangement of the airflow driving module 30 and the ultraviolet sterilization light sources 20 may have different changes. Take FIG. 4 for illustration. In another embodiment, the airflow driving module 30 is in the air channel portion 11 and located on a downwind side (the ultraviolet sterilization light sources 20 are closer to the air outlet portion 13 while the airflow driving module 30 is located closer to the air inlet portion 12) of the ultraviolet sterilization light sources 20. Take FIG. 5 for illustration. In still another embodiment, the airflow driving module 30 is in the air inlet portion 12. Since the airflow driving module 30 does not occupy space in the air channel portion 11, the number of ultraviolet sterilization light source 20 in the air channel portion 11 can become more, for example, 7. Take FIG. 6 for illustration. In still another embodiment, the airflow driving module 30 is in the air outlet portion 13. The number of the ultraviolet sterilization light source 20 in air channel portion 11 can also be increased. The airflow driving module 30 is initiated to generate an airflow from the at least one air inlet 122 to SP20-000 the at least one air outlet 132 through the air channel portion 11. In practice, airflow driving module 30 can use a centrifugal blower, an axial blower, a crossflow blower or a combination of several fans.

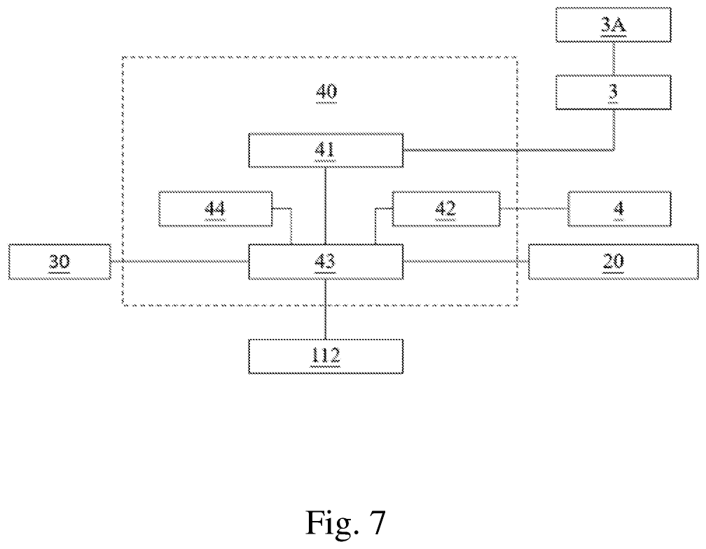

The control module 40 is electrically connected to the illuminating light 112, the ultraviolet sterilization light sources 20 and the airflow driving module 30. Its function is to control on and off of the electrically connected components. Please refer to FIG. 7. It is a circuit diagram of the control module 40 (shown by the dotted frame) of the intelligent full-time sterilization lighting device 1. The control module 40 includes a power conversion unit 41, an operating interface 42, a control unit 43 and a communication unit 44. The power conversion unit 41 is electrically connected to a power switch 3 externally. The power switch 3 is connected to a power grid 3A to get AC power from the power grid 3A. When the power switch 3 is turned on, AC power from the power switch 3 is received by the power conversion unit 41 and voltage of the AC power is dropped and converted to obtain a working power. The operating interface 42 is used to connect to an operating device 4, such as a wire control box, a wireless remote control, a mobile communication device, or even a smart phone with APP, externally. Therefore, the operating interface 42 may be a USB connector, a RJ10 connector, a RJ45 connector, an infrared signal transceiver module or a mobile communication module (3G, 4G or 5G).

The control unit 43 is electrically connected to the power conversion unit 41, illuminating light 112, the ultraviolet sterilization light sources 20 and the airflow driving module 30, signally connected to the operating interface 42. The control unit 43 can execute the following operations: A. receiving a long-time and full-field operating signal from the operating device 4 so as to keep turning on the illuminating light 112, the ultraviolet sterilization light sources 20 and the airflow driving module 30; and B. receiving a short-time and half-field operating signal from the operating device 4 so as to turn off the illuminating light 112, and keep turning on the ultraviolet sterilization light sources 20 and the airflow driving module 30 at least one operating time. Operation A is applied to a closed room where there are crowd activities and lighting is required. At this time, disinfection and sterilization work. The time can be unlimited, but it needs to consume electricity. Operation B is applied to a closed room where there is no crowd, but disinfection is required. At this time, the illuminating light 112 has been extinguished, and the ultraviolet sterilization light sources 20 and airflow driving module 30 continue to operate in the external casing 10. Of course, time to work does not need to be maintained. It will consume unnecessary electricity. Hence, is a basic sterilization time, such as one hour.

The communication unit 44 is signally connected to the control unit 43. The communication unit 44 is able to signally connect to communication units in other intelligent full-time sterilization lighting devices to transmit operating signals from the operating device 4 to signally connected intelligent full-time sterilization lighting devices. In addition, the communication unit 44 may also be designed to synchronize operations of all signally connected intelligent full-time sterilization lighting devices, for example, to set when to perform operation A and when to perform operation B. The structure of the communication unit 44 is quite simple. It may be a power line contact. Signal connection among the intelligent full-time sterilization lighting devices are accomplished by the power line between the communication units 44. The signal of the communication unit 44 can be presented by the level of potential or the change of frequency.

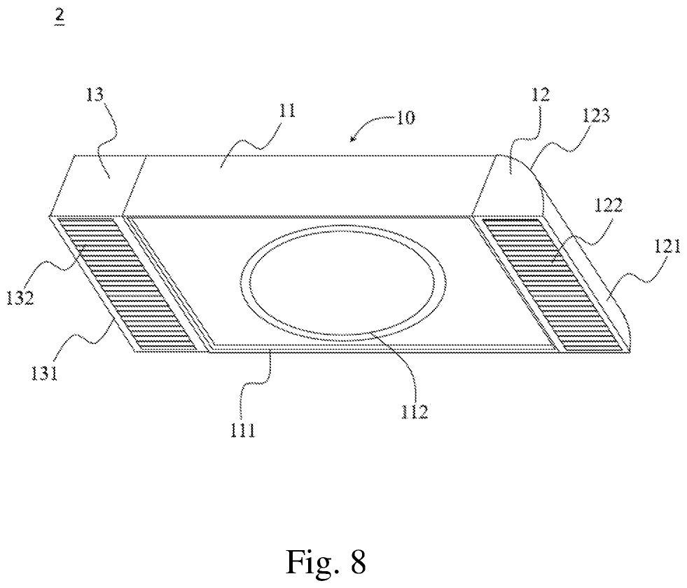

Please see FIG. 8. the Fig. is a schematic diagram of the appearance of another intelligent full-time sterilization lighting device 2 according to an embodiment of the present invention. In order to simplify the description, the same symbols used in FIG. 8 as those in FIG. 1 have the same functions and names. Comparing the intelligent full-time sterilization lighting device 1 and the intelligent full-time sterilization lighting device 2, it is found that the latter has the following differences. First, the external casing 10 is rectangular. In practice, the external casing 10 may be a rectangle with dimensions of 60 cm×120 cm to meet the size of the calcium silicate board of the current light steel frame ceiling. Of course, there are other rectangular sizes, which are not limited by the present invention. Second, the air outlet portion 13 doesn't have the curved segment 133, which is replaced by a 90-degree structure. Although this approach will increase noise, its production cost can be reduced. Third, there are only one the air inlet 122 and only one air outlet 132, but their area is quite large. In addition to reducing production costs, this method also facilitates the removal and cleaning of the air filter on it. However, appearance may not look good. Fourth, the illuminating light 112 uses a single circular fluorescent lamp.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims, which are to be accorded with the broadest interpretation so as to encompass all such modifications and similar structures.

What is claimed is:

1. An intelligent full-time sterilization lighting device, comprising:
an external casing, formed of an air channel portion and an air inlet portion and an air outlet portion on both sides thereof, wherein a lighting zone is formed below an outside of the air channel portion, and two sides of the lighting zone in correspondence to the air inlet portion and the air outlet portion form an air inlet zone and an air outlet zone, respectively, wherein at least one illuminating light is installed in the lighting zone, the air inlet zone has at least one air inlet, the at least one air inlet is equipped with an air filter to filter floating dust in the external air, the air outlet zone has at least one air outlet, the at least one air outlet is equipped with an air filter to filter floating dust in the air flowing through the external casing, and an angle between a normal direction of the lighting zone and a normal direction of the at least one air inlet and an angle between the normal direction of the lighting zone and a normal direction of the at least one air outlet are not larger than a first angle;
a plurality of ultraviolet sterilization light sources, installed in the air channel portion;
an airflow driving module, installed on any location of the air channel portion, the air inlet portion or the air outlet portion, initiated to generate an airflow from the at least one air inlet to the at least one air outlet through the air channel portion; and
a control module, electrically connected to the illuminating light, the ultraviolet sterilization light sources and the airflow driving module, controlling on and off of the electrically connected components.

2. The intelligent full-time sterilization lighting device according to claim 1, wherein the first angle is 10 degrees.

3. The intelligent full-time sterilization lighting device according to claim 1, wherein the air inlet portion forms a curved segment, connected to the air channel portion and the at least one air inlet to reduce noise when airflow passes by.

4. The intelligent full-time sterilization lighting device according to claim 1, wherein the air outlet portion forms a curved segment, connected to the air channel portion and the at least one air outlet to reduce noise when airflow passes by.

5. The intelligent full-time sterilization lighting device according to claim 1, wherein the ultraviolet sterilization light source is an ultraviolet LED light bar, a strip ultraviolet sterilization lamp or a circular ultraviolet sterilization lamp.

6. The intelligent full-time sterilization lighting device according to claim 1, wherein the control module further comprises:
a power conversion unit, electrically connected to a power switch externally, wherein when the power switch is on, AC power from the power switch is received and voltage thereof is dropped and converted to obtain a working power;
an operating interface, connecting to an operating device externally; and
a control unit, electrically connected to the power conversion unit, the illuminating light, the ultraviolet sterilization light sources and the airflow driving module and signally connected to the operating interface, executing the following operations:
receiving a long-time and full-field operating signal from the operating device so as to keep turning on the illuminating light, the ultraviolet sterilization light sources and the airflow driving module; and
receiving a short-time and half-field operating signal from the operating device so as to turn off the illuminating light, and keep turning on the ultraviolet sterilization light sources and the airflow driving module at least one operating time.

7. The intelligent full-time sterilization lighting device according to claim 6, wherein the operating time is one hour.

8. The intelligent full-time sterilization lighting device according to claim 6, wherein the control module further comprises a communication unit, signally connected to the control unit, wherein the communication unit is able to signally connect to communication units in other intelligent full-time sterilization lighting devices to transmit operating signals from the operating device to signally connected intelligent full-time sterilization lighting devices, or synchronize operations of all signally connected intelligent full-time sterilization lighting devices.

9. The intelligent full-time sterilization lighting device according to claim 6, wherein the operating interface is a USB connector, a RJ10 connector, a RJ45 connector, an infrared signal transceiver module or a mobile communication module.

10. The intelligent full-time sterilization lighting device according to claim 6, wherein the communication unit is a power line contact.

11. The intelligent full-time sterilization lighting device according to claim 1, wherein the ultraviolet sterilization light sources are closer to the air outlet portion while the airflow driving module is closer to the air inlet portion.

12. The intelligent full-time sterilization lighting device according to claim 1, wherein the airflow driving module is a centrifugal blower, an axial blower, a crossflow blower or a combination of a plurality of fans.
